(19) United States
(12) Patent Application Publication
(10) Pub. No.: US 2018/0311134 A1
(43) Pub. Date: Nov. 1, 2018

(54) ODORLESS THIOLS FOR PERMANENT WAVING, STRAIGHTENING AND DEPILATORY APPLICATIONS

(71) Applicant: The Procter & Gamble Company, Cincinnati, OH (US)

(72) Inventors: Guiru Zhang, Lebanon, OH (US); Stephanie Lee Davis, Liberty Township, OH (US); Bryan Patrick Murphy, Loveland, OH (US); David Salloum, West Chester, OH (US)

(21) Appl. No.: 15/498,566

(22) Filed: Apr. 27, 2017

(51) Int. Cl. A61K 8/49; A61K 8/22; A61K 8/19; A61Q 5/06; A61Q 9/04; A45D 7/04

(52) U.S. Cl. CPC A61K 8/4933; A61K 8/22; A61K 8/19; A61K 2800/884; A61Q 9/04; A45D 7/04; A61Q 5/06

(57) ABSTRACT

Described herein is a method for treating hair including: (a) putting the hair in a desired shape; (b) before and/or after the hair is put in the desired shape, applying a reducing composition to the hair including: (i) from about 2.5% to about 15% of a cationic mercapto amino pyridinium compound or a cationic dimercapto amino pyridinium compound; (ii) from about 4% to about 10% of a buffering system; and (iii) from about 50% to about 93.5% of a solvent; (c) rinsing the reducing composition from the hair; (d) applying an oxidizing composition to the hair including: (i) from about 0.5% to about 12% of an oxidizing agent; and (ii) from about 80% to about 97% of a solvent; and (e) rinsing the oxidizing composition from the hair. The reducing composition has a pH of from about 8 to about 10.5;

### FIELD OF THE INVENTION

Described herein is the design and synthesis of a class of cationic thiols for permanent shaping, waving, straightening, and depilatory applications.

### BACKGROUND OF THE INVENTION

The permanent shaping and/or alteration of the curvature of keratinous fibers, in particular human hair, by the application of ammonium thioglycolate (perm salt), or sodium thiogylcolate, is known. In order to provide the consumer with the desired waved or straightened hair, an alkaline solution of perm salt is often utilized. Permanent waving formulations typically comprise perm salt and alkalizers. Thioglycolate enters the hair shaft and reduces the —S—S— disulfide bond, thus allowing hair fibers to relax from their original shape. After hair is set to its desired new wavy patterns or straightened, a formulation containing hydrogen peroxide is then applied to reform the disulfide bonds and oxidize unreacted thioglycolate.

Permanent waving and straightening products are typically sold in the form of kits containing a relaxing component, e.g., an alkaline solution of reducing agent, and an oxidizing component, e.g., a hydrogen peroxide solution. In use, the relaxing component is first applied to hair to either create wave patterns or straighten hair by breaking the disulfide bonds in keratin proteins. After waiting a certain amount of time for the perm salt to do the chemistry, hair is rinsed then an oxidizing cream or gel is applied to reform a good portion of the disulfide bonds and thus fix the new curvature or newly straightened hair.

Chemical depilation is typically done at high pH with a reducing agent, typically thioglycolate, to break the disulfide bond. The high pH cream or gel is typically applied directly on skin where undesirable hair needs to be removed. After a certain amount of time, the cream/gel, together with degraded hair, is wiped off skin. Next, skin is rinsed to complete the process. The same technology can be used in shaving cream to help soften facial hair, which makes subsequent shaving go easier and smoother.

Since Arnold F. Willatt first invented the cold perm using thioglycolate in 1938, many attempts have been made by the perming industry to reduce or eliminate the offensive odor associated with the otherwise very effective treatment. Masking with fragrance is typically done with all products containing thioglycolate and derivatives. However, it cannot completely mask the odor, especially post treatment. Modifying the thioglycolate to reduce its offensive odor has also been tried. Thiols with higher molecular weight generally are less volatile, thus less of it reaches the olfactory cells. However, it would also be more difficult for a bigger molecule to penetrate hair to remain effective. There is also the corresponding issue on formulating a less polar ingredient in aqueous based chassis. One of the ways to address the polarity issue is to add polar groups to the molecule to make it more water soluble. One of the adverse effects for high polarity is lower partition in hair. With lower partition, the efficiency of the thiol drops.

Thiols carrying an aliphatic quaternary ammonium cation have also been explored to minimize the sulfurous stench. They are stable at acidic and neutral conditions. Under in-use conditions at high pH, however, these thiols would degrade to generate a tertiary amine and a cyclic sulfide, which defeats the purpose of an odor free technology.

There have also been attempts to use cross-linking reagents without the thiol alcohol functionality to replace thioglycolate. The fact that Brazilian perm uses up to 10 wt. % formaldehyde to relax curly hair highlights the desperation of the penning and straightening industry to disassociate itself from the pungent stench of thioglycolate salts.

Accordingly, there is a need for a solution to using thiols to create desirable hair styles without the undesirable in-use experience, including odor.

### SUMMARY OF THE INVENTION

Described herein is a method for treating hair comprising: (a) putting the hair in a desired shape; (b) before and/or after the hair is put in the desired shape, applying a reducing composition to the hair comprising: (i) from about 2.5% to about 15% of a cationic mercapto amino pyridinium compound or a cationic dimercapto amino pyridinium compound, by weight of the reducing composition; (ii) from about 4% to about 10% of a buffering system, by weight of the reducing composition; and (iii) from about 50% to about 93.5% of a solvent, by weight of the reducing composition; wherein the reducing composition has a pH of from about 8 to about 10.5; (c) rinsing the reducing composition from the hair; (d) applying an oxidizing composition to the hair comprising: (i) from about 0.5% to about 12% of an oxidizing agent, by weight of the oxidizing composition; and (ii) from about 80% to about 97% of a solvent, by weight of the oxidizing composition; and (e) rinsing the oxidizing composition from the hair.

### DETAILED DESCRIPTION OF THE INVENTION

While the specification concludes with claims which particularly point out and distinctly claim the invention, it is believed the present invention will be better understood from the following description.

All percentages, parts and ratios are based upon the total weight of the compositions of the present invention, unless otherwise specified. All such weights as they pertain to listed ingredients are based on the active level and, therefore, do not include solvents or by-products that may be included in commercially available materials, unless otherwise specified. When more than one composition is used during a treatment, as in mixing of the components of a typical perming/straightening product, the total weight to be considered is the total weight of all the compositions applied on the hair simultaneously (i.e. the weight found “on head”) unless otherwise specified. The term “weight percent” may be denoted as “wt. %” herein.

As used herein, the term “hair” to be treated may be “living” i.e. on a living body or may be “non-living” i.e. in a wig, hairpiece or other aggregation of non-living keratinous fibers. Mammalian, particularly human, hair is preferred. However, wool, fur, and other keratin containing fibers are suitable substrates for the compositions according to the present invention.

As used herein, the term “thiols” means mercapto amino pyridinium compounds or dimercapto amino pyridinium compounds.

The hair permanent waving or straightening compositions of the present invention comprise one or more cationic thiols.

Cationic thiols, as well as other relevant components, are described in detail hereinafter.

### I. Odorless Cationic Thiols

Described herein is a general method to derive odorless cationic thiols according to the formulas defined herein. Also described herein are methods and compositions for the reshaping of keratin fibers comprising at least one cationic thiol according to the formulas defined herein. Also described herein are methods comprising applying such compositions to the keratin fiber, followed by an oxidizing agent, for a period of time sufficient to shape the hair and develop the desirable hair style.

It is understood that numerous potentially and actually tautomeric compounds are involved. Thus, for example, 2-mercaptopyridine (I) exists under known conditions in the pyridine-2-thione tautomer form (II).

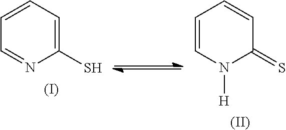

It is to be understood that when this development refers to a particular structure, all of the reasonable additional tautomeric structures are included. In the art, tautomeric structures are frequently represented by one single structure and the disclosure herein follows this general practice.

As used herein, the term “thiols” means mercapto amino pyridinium compounds or dimercapto amino pyridinium compounds. Here, an aromatic cation is attached to a thiol through a linker group to address the offensive odor associated with thiols by converting thiols into organic salts with extremely low volatility. Since these molecules generally won't be present in air, they can't be detected by olfactory cells during and after treatment. The cationic thiol would typically carry one permanent positive charge such as pyridinium, imidazolium, thiazolium, or an analogous cation under basic conditions for typical permanent waving, straightening and depilatory applications.

There are additional benefits associated with turning thiols into zwitterionic or cationic species under in-use conditions. Hair deposition/penetration can be greatly enhanced compared to TGA (thioglycolic acid), which is anionic under in-use conditions. So the efficiency goes up for thiols with a cationic center(s). Penetration into live skin cells, however, would drop as the cell membrane, a lipid bilayer, would only allow a certain cation and anion to go through the ion channels and keep most other salts out.

Previous attempts to link a thiol to an aliphatic quaternary ammonium cation could not deliver an odorless technology at high pH, which is required for the deprotonation of the thiol to make it nucleophilic enough to break disulfide bond efficiently. They can be stable and odorless under acidic and neutral conditions. At high pH, however, compound (III) can be deprotonated to yield an unstable intermediate (IV), which can undergo a decomposition reaction to produce a tertiary amine (V) and a cyclic sulfide (VI), which can both carry unpleasant odors. The conversion rate of the degradation does not have to be high before the olfactory cells pick up the odor.

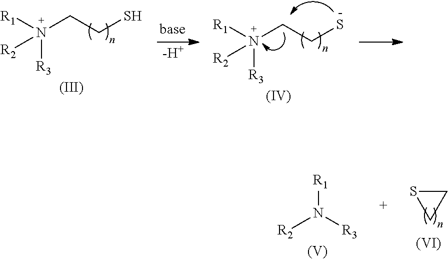

Similar to the above case, not all thiols linked to an aromatic cation are stable, either. Compound (VII) can be stable under acidic and neutral conditions. Under basic conditions, however, it can undergo a degradation reaction through intermediate (VIII) to provide (IX) and thiirane, which can make it an unsuitable candidate for the odor free technology.

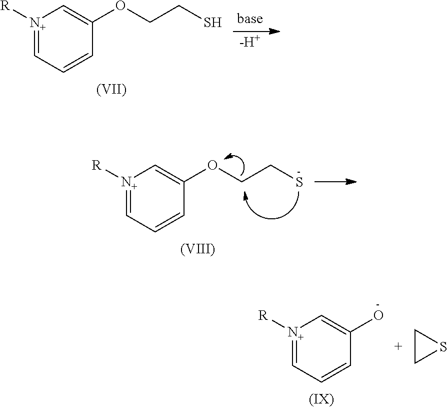

The odorless cationic thiols described herein differ from previous approaches in terms of high pH stability and undetectable level of odor. The thiols described herein carrying an aromatic cation can be extremely stable at high pH. They can also be efficient in breaking the disulfide bonds in keratin fibers. Some of them are 3 times more efficient than thioglycolate salts—therefore substantially equal performance in perming can be achieved with ⅓ molar concentration of the active ingredient compared to the benchmark ammonium thioglycolate. In addition, the cationic thiols can be water soluble as organic salts, which makes it convenient to formulate in an aqueous based chassis. Last, the cationic thiols can pass the odor test in formulation and during on hair applications as stench free.

In an embodiments, the cationic thiol is of formula (Xa);

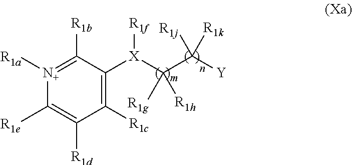

wherein

R₁ₐ is linear, branched or cyclo alkyl, aminoalkyl, hydroxyalkyl or alkenyl; and

R_{1b}, R_{1c}, R_{1d} and R₁ₑ are each independently hydrogen, alkyl, alkenyl, alkynyl, aryl, hydroxyalkyl, aminoalkyl, acyl, or a heterocyclic moiety; and

R_{1f} is hydrogen, alkyl, alkenyl, hydroxyl alkyl or amino alkyl; and
R_{1g}, R₁ₕ, R₁ⱼ and R₁ₖ are each independently hydrogen, alkyl, alkenyl, alkynyl, aryl, hydroxyalkyl, or aminoalkyl; and

m and n are independently whole numbers ranging from 0 to 5; and

X is CH or N. When X is a carbon atom, m+n=0, when X is a nitrogen atom, m+n≥2; and

Y is SH or isothiouronium salt.

In an embodiment, the cationic thiol is of formula (Xb);

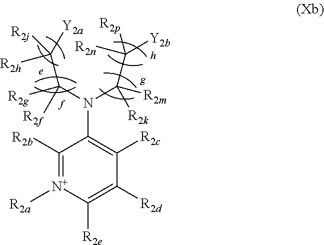

wherein

R₂ₐ is linear, branched or cyclo alkyl, aminoalkyl, hydroxyalkyl or alkenyl; and

R_{2b}, R_{2c}, R₂ₐ and R₂ₑ are each independently hydrogen, alkyl, alkenyl, alkynyl, aryl, hydroxyalkyl, aminoalkyl, acyl, or a heterocyclic moiety; and

R_{2f}, R_{2g}, R₂ₕ, R₂ⱼ, R₂ₖ, R₂ₘ, R₂ₙ and R₂ₚ are each independently hydrogen, alkyl, alkenyl, alkynyl, aryl, hydroxyalkyl, or aminoalkyl; and

e, f, g and h are independently whole numbers ranging from 0 to 5. However, the four numbers much satisfy the following equations: e+f≥2; g+h≥2; and

Y₂ₐ and Y_{2b} are each independently SH or isothiouronium salt.

In an embodiment, the cationic thiol is of formula (Xc);

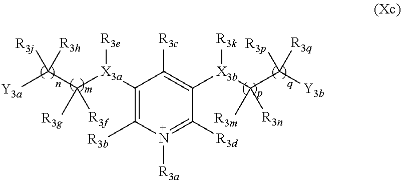

wherein

R₁ₐ is linear, branched or cyclo alkyl, aminoalkyl, hydroxyalkyl or alkenyl; and

R_{3b}, R_{3c} and R_{3d} are each independently hydrogen, alkyl, alkenyl, alkynyl, aryl, hydroxyalkyl, aminoalkyl, acyl, or a heterocyclic moiety; and

R₃ₑ and R₃ₖ are each independently hydrogen, alkyl, alkenyl, hydroxyl alkyl or amino alkyl; and

R_{3f}, R_{3g}, R₃ₕ, R₃ⱼ, R₃ₘ, R₃ₙ, R₃ₚ, and R_{3q} are each independently hydrogen, alkyl, alkenyl, alkynyl, aryl, hydroxyalkyl, or aminoalkyl; and

m, n, p and q are independently whole numbers ranging from 0 to 5; and

X₃ₐ and X_{3b} are each independently CH or N. When X₃ₐ is CH, m+n=0, when X₃ₐ is N, m+n≥2; When X_{3b} is CH, p+q=0, when X_{3b} is N, p+q≥2; and

Y₃ₐ and Y_{3b} are each independently SH or isothiouronium salt.

In other embodiments, the cationic thiol is of formula (Xd);

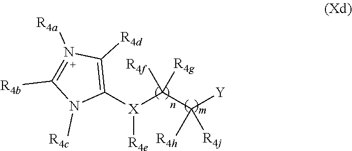

wherein

R₄ₐ is linear, branched or cyclo alkyl, aminoalkyl, hydroxyalkyl or alkenyl; and

R_{4b}, R_{4c} and R_{4d} are each independently hydrogen, alkyl, alkenyl, alkynyl, aryl, hydroxyalkyl, aminoalkyl, acyl, or a heterocyclic moiety; and

R₄ₑ is hydrogen, alkyl, alkenyl, hydroxyl alkyl or amino alkyl; and

R_{4f}, R_{4g}, R₄ₕ and R₄ⱼ are each independently hydrogen, alkyl, alkenyl, alkynyl, aryl, hydroxyalkyl, or aminoalkyl; and

m and n are independently whole numbers ranging from 0 to 5; and

X is CH or N. When X is CH, m+n=0, when X is N, m+n≥2; and

Y is thiol or isothiouronium salt.

In other embodiments, the cationic thiol is of formula (Xe);

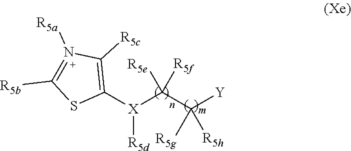

wherein

R₅ₐ is linear, branched or cyclo alkyl, aminoalkyl, hydroxyalkyl or alkenyl; and

R_{5b} and R_{5c} are each independently hydrogen, alkyl, alkenyl, alkynyl, aryl, hydroxyalkyl, aminoalkyl, acyl, or a heterocyclic moiety; and

R_{5d} is hydrogen, alkyl, alkenyl, hydroxyl alkyl or amino alkyl; and

R₅ₑ, R_{5f}, R_{5g} and R₅ₕ are each independently hydrogen, alkyl, alkenyl, alkynyl, aryl, hydroxyalkyl, or aminoalkyl; and

m and n are independently whole numbers ranging from 0 to 5; and

X is CH or N. When X is CH, m+n=0, when X is N, m+n≥2; and

Y is thiol or isothiouronium salt.

In other embodiments, the cationic thiol is of formula (Xf);

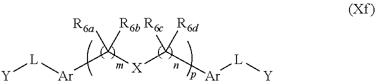

wherein

Ar is a positively charged aromatic ring such as pyridinium, imidazolium or thiazolium; and

X is CR₆ₑR_{6f}, NR_{6g}, O or S; and

Y is thiol or isothiouronium salt; and

L is a linker group connecting Y and Ar.

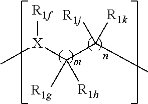

as defined in Xa

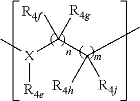

as defined in Xd

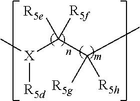

as defined in Xe

It can be the above moieties as defined in Xa, Xd or Xe; and

R₆ₐ, R_{6b}, R_{6c}, R_{6d}, R₆ₑ, R_{6f} and R_{6g} are each independently hydrogen, alkyl, alkenyl, alkynyl, aryl, hydroxyalkyl, or aminoalkyl; and

p is a whole number ranging from 1 to 5. m and n are independently whole numbers ranging from 1 to 6.

In other embodiments, the cationic compound is of formula (Xg);

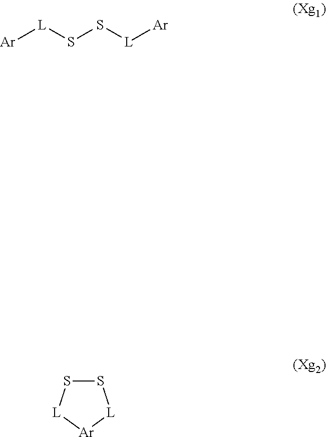

wherein

Ar is a positively charged aromatic ring such as pyridinium, imidazolium or thiazolium; and

L is a linker group connecting the disulfide functionality and Ar. L is defined in the same way as in Xf.

### Exemplary Synthesis

### Synthesis of 3-(mercaptomethyl)-1-methylpyridin-1-ium trifluoroacetate

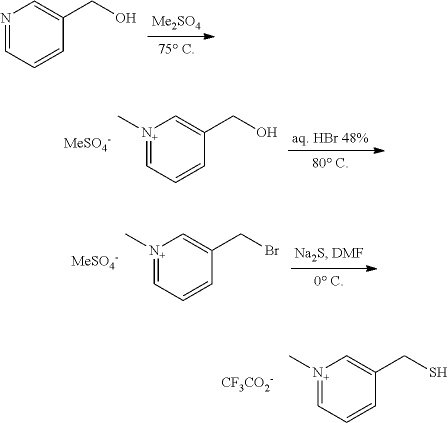

To pyridin-3-ylmethanol (2.00 g) cooled in ice-water bath in a tall scintillation vial equipped with silicone septa is added neat dimethylsulfate (2.43 g). The reaction mixture is then sealed with screw cap and allowed to warm up to room temperature and go beyond while magnetically stirred. After the heat rise ceases and the temperature of the reaction mixture drops back to ambient, the reaction mixture is heated to 75° C. on oil bath for an additional hour while magnetically stirred. The resulting thick oil is purified on automated flash column chromatography on silica with dichloromethane and methanol as mobile phase to provide 3-(hydroxymethyl)-1-methylpyridin-1-ium methylsulfate (4.10 g) as thick colorless oil in 95% yield.

To 3-(hydroxymethyl)-1-methylpyridin-1-ium methylsulfate (4.10 g) in a 500 mL round bottom flask is added aqueous HBr (48%, 150 mL). The reaction mixture is then heated to 80° C. while magnetically stirred. Once LCMS confirms the complete conversion of the alcohol to corresponding bromide, excess aqueous HBr is then removed in vacuo. The residual reddish brown oil is purified on automated flash column chromatography on silica with dichloromethane and methanol as mobile phase to provide 3-(bromomethyl)-1-methylpyridin-1-ium methylsulfate (4.31 g) as thick amber colored oil in 83% yield.

To 3-(bromomethyl)-1-methylpyridin-1-ium methylsulfate (1.00 g) in a 100 mL evaporating flask is added DMF (anhydrous, 30 mL). The solution is then cooled in an ice-water bath. To the chilled solution is then added sodium sulfide (0.31 g). The reaction mixture is then allowed to warm up to room temperature gradually while being magnetically stirred. The color of the solution turns dark as the reaction proceeds. Once HPLC confirms that the conversion of the bromide to corresponding mercapto compound has stalled, the reaction is then quenched with 1 N HCl (6 mL). Solvents are then removed in vacuo. The residual dark brown slurry is purified on automated flash column chromatography on C-18 reverse phase column with water and acetonitrile with 0.1% TFA as mobile phase to provide 3-(mercaptomethyl)-1-methylpyridin-1-ium trifluoroacetate (0.56 g) as white solid in 66% yield.

### Synthesis of 3-((2-mercaptoethyl)amino)-1-methylpyridin-1-ium trifluoroacetate

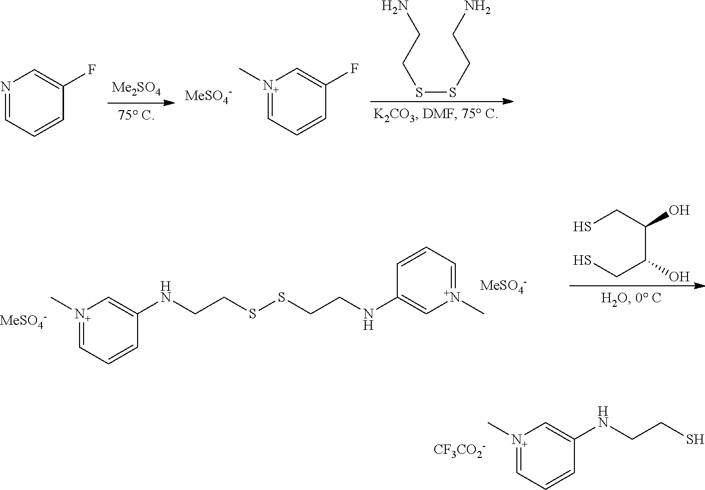

To 3-fluoropyridine (3.00 g) in a tall scintillation vial equipped with silicone septa is added neat dimethylsulfate (4.09 g). The vial is then sealed and magnetically stirred. After the heat rise ceases and the temperature of the reaction mixture drops back to room temperature, the reaction mixture is heated to 75° C. on oil bath for an additional hour while magnetically stirred. The resulting thick oil is purified on automated flash column chromatography on silica with dichloromethane and methanol as mobile phase to provide 3-fluoro-1-methylpyridin-1-ium methylsulfate (6.41 g) as thick colorless oil in 93% yield.

To 3-fluoro-1-methylpyridin-1-ium methylsulfate (1.50 g) dissolved in DMF (10 mL) in a tall scintillation vial equipped with silicone septa is added potassium carbonate (1.00 g) and 2,2′-dithiobis-ethanamine (0.47 g). The reaction mixture is then heated at 75° C. for 3 hours while magnetically stirred. Once the conversion is confirmed to be complete by UPLC, potassium salts are filtered off and DMF removed in vacuo. The residual reaction mixture is then purified by automated flash column chromatography on silica with DCM and MeOH as mobile phase to provide 3,3′-((disulfanediylbis(ethane-2,1-diyl))bis(azanediyl))bis(1-methylpyridin-1-ium) methylsulfate (1.55 g) as a pale yellowish solid in 91% yield.

To cooled 3,3′-((disulfanediylbis(ethane-2,1-diyl))bis(azanediyl))bis(1-methylpyridin-1-ium) methylsulfate (1.00 g) dissolved in water (10 mL) in a tall scintillation vial equipped with silicone septa is added DL-1,4-dithiothreitol (0.33 g). The reaction mixture is magnetically stirred in ice-water bath for 1 hour. Once the conversion is confirmed to be complete by UPLC, the reaction mixture is then purified by automated flash column chromatography on C-18 reverse phase column with MeOH/H₂O with 0.1% TFA as mobile phase to provide 3-((2-mercaptoethyl)amino)-1-methylpyridin-1-ium trifluoroacetate (0.80 g) as a pale yellowish solid in 79% yield.

### Synthesis of 3-[bis(2-mercaptoethyl)amino]-1-methylpyridin-1-ium trifluoroacetate

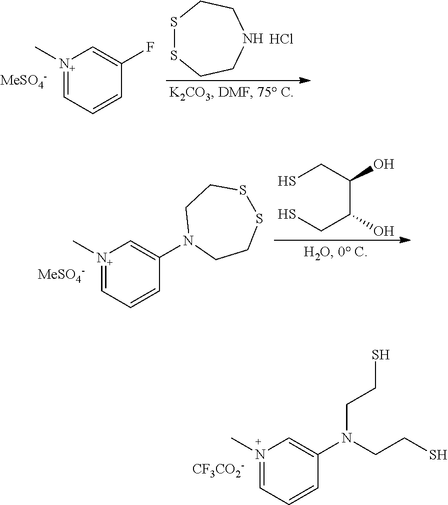

To 3-fluoro-1-methylpyridin-1-ium methylsulfate (1.56 g) dissolved in DMF (10 mL) in a tall scintillation vial equipped with silicone septa is added potassium carbonate (1.50 g) and 1,2,5-dithiazepane hydrochloride (1.00 g). The reaction mixture is then heated at 75° C. for 2 hours while magnetically stirred. Once the conversion is confirmed to be complete by UPLC, potassium salts are filtered off and DMF removed in vacuo. The residual reaction mixture is then purified by automated flash column chromatography on silica with DCM and MeOH as mobile phase to provide 3-(1,2,5-dithiazepan-5-yl)-1-methylpyridin-1-ium methylsulfate (1.62 g) as a pale yellowish solid in 82% yield.

To cooled 3-(1,2,5-dithiazepan-5-yl)-1-methylpyridin-1-ium methylsulfate (1.00 g) dissolved in water (15 mL) in a tall scintillation vial equipped with silicone septa is added DL-1,4-dithiothreitol (0.60 g). The reaction mixture is magnetically stirred in ice-water bath for 1 hour. Once the conversion is confirmed to be complete by UPLC, the reaction mixture is then purified by automated flash column chromatography on C-18 reverse phase column with MeOH/H₂O with 0.1% TFA as mobile phase to provide 3-[bis(2-mercaptoethyl)amino]-1-methylpyridin-1-ium trifluoroacetate (0.74 g) as a pale yellowish solid in 73% yield.

### Synthesis of 3-((2-((amino(iminio)methyl)thio)ethyl)amino)-1-methylpyridin-1-ium trifluoroacetate

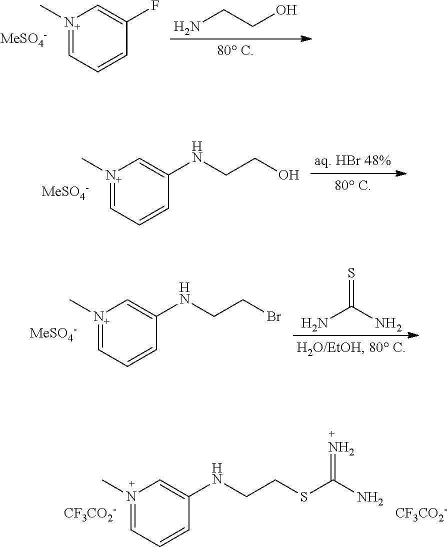

To 3-fluoro-1-methylpyridin-1-ium methylsulfate (3.00 g) in a tall scintillation vial equipped with silicone septa is added neat ethanolamine (2.05 g). The reaction mixture is then heated at 80° C. for 3 hours while magnetically stirred. Once the conversion is confirmed to be complete by UPLC, the reaction mixture is then purified by automated flash column chromatography on silica with DCM and MeOH as mobile phase to provide 3-((2-hydroxyethyl)amino)-1-methylpyridin-1-ium methylsulfate (3.41 g) as thick colorless oil in 96% yield.

To 3-((2-hydroxyethyl)amino)-1-methylpyridin-1-ium methylsulfate (3.41 g) in a 500 mL round bottom flask is added aqueous HBr (48%, 150 mL). The reaction mixture is then heated to 80° C. while magnetically stirred. Once LCMS confirms the complete conversion of the alcohol to corresponding bromide, excess aqueous HBr is then removed in vacuo. The residual reddish brown oil is purified on automated flash column chromatography on silica with dichloromethane and methanol as mobile phase to provide 3-((2-bromoethyl)amino)-1-methylpyridin-1-ium methylsulfate (3.63 g) as thick amber colored oil in 86% yield.

To 3-((2-bromoethyl)amino)-1-methylpyridin-1-ium methylsulfate (1.00 g) dissolved in water/EtOH (v/v=1/1, 20 mL) in a tall scintillation vial equipped with silicone septa is added thiourea (0.28 g). The reaction mixture is then heated in oil bath to 80° C. while magnetically stirred for 1 hour. Once the conversion is confirmed to be complete by UPLC, solvents are then removed in vacuo, the residual reaction mixture is then purified by automated flash column chromatography on C-18 reverse phase column with MeOH/H₂O with 0.1% TFA as mobile phase to provide 3-((2-((amino(iminio)methyl)thio)ethyl)amino)-1-methylpyridin-1-ium trifluoroacetate (1.23 g) as a pale yellowish solid in 92% yield.

### Synthesis of 1,1′-(butane-1,4-diyl)bis(3-(mercaptomethyl)pyridin-1-ium) bromide

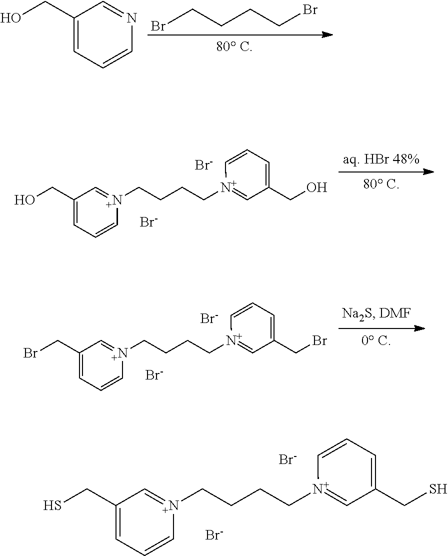

To pyridin-3-ylmethanol (3.00 g) in a tall scintillation vial equipped with silicone septa is added neat 1,4-dibromobutane (2.00 g). The reaction mixture is then sealed with screw cap and heated to 80° C. on oil bath for overnight while magnetically stirred. The resulting solid is purified on automated flash column chromatography on silica with dichloromethane and methanol as mobile phase to provide 1,1′-(butane-1,4-diyl)bis(3-(hydroxymethyl)pyridin-1-ium) bromide (3.58 g) as white solid in 89% yield.

To 1,1′-(butane-1,4-diyl)bis(3-(hydroxymethyl)pyridin-1-ium) bromide (3.00 g) in a 250 mL round bottom flask is added aqueous HBr (48%, 100 mL). The reaction mixture is then heated to 80° C. while magnetically stirred. Once LCMS confirms the complete conversion of the alcohol to corresponding bromide, excess aqueous HBr is then removed in vacuo. The residual reddish brown oil is purified on automated flash column chromatography on silica with dichloromethane and methanol as mobile phase to provide 1,1′-(butane-1,4-diyl)bis(3-(bromomethyl)pyridin-1-ium) bromide (3.29 g) as thick amber colored solid in 85% yield.

To 1,1′-(butane-1,4-diyl)bis(3-(bromomethyl)pyridin-1-ium) bromide (1.50 g) in a 100 mL evaporating flask is added DMF (anhydrous, 30 mL). The solution is then cooled in an ice-water bath. To the chilled solution is then added sodium sulfide (0.84 g). The reaction mixture is then allowed to warm up to room temperature gradually while being magnetically stirred. The color of the solution turns dark as the reaction proceeds. Once HPLC confirms that the conversion of the bromide to corresponding mercapto compound has stalled, the reaction is then quenched with 1 N HBr (12 mL). Solvents are then removed in vacuo. The residual dark brown slurry is purified on automated flash column chromatography on C-18 reverse phase column with water and acetonitrile as mobile phase to provide 1,1′-(butane-1,4-diyl)bis(3-(mercaptomethyl)pyridin-1-ium) bromide (0.72 g) as white solid in 58% yield.

### II. Reducing Composition

Described herein is a method for treating hair comprising: (a) putting the hair in a desired shape; (b) before and/or after the hair is put in the desired shape, applying a reducing composition to the hair; (c) rinsing the reducing composition from the hair; (d) applying an oxidizing composition to the hair; and (e) rinsing the oxidizing composition from the hair.

The reducing composition can comprise from about 2% to about 20%, alternatively from about 2% to about 18%, alternative from about 2.5% to about 15%, alternatively from about 5% to about 13%, alternatively from about 7% to about 12%, and alternatively from about 9% to about 11% of a cationic mercapto amino pyridinium compound or a cationic dimercapto amino pyridinium compound, by weight of the reducing composition. The reducing composition can comprise only the cationic mercapto amino pyridinium compound. The cationic mercapto amino pyridinium compound can be 3-((2-mercaptoethyl)amino)-1-methylpyridin-1-ium iodide.

The reducing composition can comprise from about 4% to about 10%, alternatively from about 5% to about 7% of a buffering system, by weight of the reducing composition. The buffering system can comprise ammonium carbonate and/or ammonium hydroxide.

Suitable pH modifiers and/or buffering agents include, but are not limited to: ammonia; alkanolamides (such as monoethanolamine, diethanolamine, triethanolamine, monopropanolamine, dipropanolamine, tripropanolamine, tripropanolamine, 2-amino-2-methyl-1-propanol, 2-amino-2-hydroxymethyl-1,3,-propandiol); guanidium salts; alkali metal and ammonium hydroxides and carbonates; and mixtures thereof.

Further pH modifiers and/or buffering agents include, but are not limited to: sodium hydroxide; ammonium carbonate; acidulents (such as inorganic and inorganic acids including for example phosphoric acid, acetic acid, ascorbic acid, citric acid or tartaric acid, hydrochloric acid); and mixtures thereof.

The reducing composition can comprise from about 50% to about 93.5%, alternatively from about 55% to about 92.5%, alternatively from about 60% to about 92.5%, alternatively from about 65% to about 92.5%, alternatively from about 70% to about 90% of a solvent, by weight of the reducing composition.

The solvent can be selected from water, or a mixture of water and at least one organic solvent to dissolve the compounds that would not typically be sufficiently soluble in water.

Suitable organic solvents include, but are not limited to: C1 to C4 lower alkanols (such as ethanol, propanol, isopropanol); aromatic alcohols (such as benzyl alcohol and phenoxyethanol); polyols and polyol ethers (such as carbitols, 2-butoxyethanol, propylene glycol, propylene glycol monomethyl ether, diethylene glycol monoethyl ether, monomethyl ether, hexylene glycol, glycerol, ethoxy glycol, butoxydiglycol, ethoxydiglycerol, dipropyleneglocol, polygylcerol); propylene carbonate; and mixtures thereof.

The solvent can also be selected from the group consisting of water, ethanol, propanol, isopropanol, glycerol, 1,2-propylene glycol, hexylene glycol, ethoxy diglycol, and mixtures thereof.

The reducing composition can have a pH of from about 7 to about 11, alternatively from about 8 to about 10.5, alternatively from about 9 to about 10.

The reducing composition can further comprise one or more optional ingredients selected from the group consisting of chelants, radical scavengers, thickeners, rheology modifiers, salt, carbonate ion sources, conditioning agents, surfactants, perfumes, and mixtures thereof.

The oxidizing composition can comprise from about 0.5% to about 12%, alternatively from about 1% to about 8%, and alternatively from about 1% to about 5% of an oxidizing agent, by weight of the oxidizing composition.

The oxidizing agent can be selected from water soluble peroxygen oxidizing agents. Water-soluble peroxygen oxidizing agents include, but are not limited to, hydrogen peroxide, inorganic alkali metal peroxides such as sodium periodate and sodium peroxide and organic peroxides such as urea peroxide, melamine peroxide, and inorganic perhydrate salt bleaching compounds, such as the alkali metal salts of perborates, percarbonates, perphosphates, persilicates, persulfates and the like. These inorganic perhydrate salts may be incorporated as monohydrates, tetrahydrates etc. Alkyl and aryl peroxides, and or peroxidases, oxidases, and uricases and their substrates may also be used.

Mixtures of two or more such oxidizing agents can also be used if desired. The oxidizing agents may be provided in aqueous solution or as a powder which is dissolved prior to use. In an embodiment, the oxidizing agents may be selected from the group consisting of hydrogen peroxide, percarbonate, persulfates and combinations thereof.

A potential oxidizing agent for use herein is a source of peroxymonocarbonate ions formed in situ from a source of hydrogen peroxide and a hydrogen carbonate ion source. Moreover, this system can be particularly effective in combination with a source of ammonia or ammonium ions. Accordingly, any source of these peroxymonocarbonate ions may be used. Suitable sources for use herein include sodium, potassium, guanidine, arginine, lithium, calcium, magnesium, barium, ammonium salts of carbonate, carbamate and hydrocarbonate ions and mixtures thereof such as sodium carbonate, sodium hydrogen carbonate, potassium carbonate, potassium hydrogen carbonate, guanidine carbonate, guanidine hydrogen carbonate, lithium carbonate, calcium carbonate, magnesium carbonate, barium carbonate, ammonium carbonate, ammonium hydrogen carbonate and mixtures thereof. Percarbonate salts may be used both as an oxidizing agent and as a source of carbonate ions. Sources of carbonate ions, carbamate and hydrocarbonate ions include sodium hydrogen carbonate, potassium hydrogen carbonate, ammonium carbamate, and mixtures thereof. In an embodiment, the oxidizing composition is selected from the group consisting of potassium bromate, sodium bromate, sodium perborate, dehydroascorbic acid, hydrogen peroxide, urea peroxide, and mixtures thereof. In an embodiment, the oxidizing agent can be hydrogen peroxide.

The oxidizing composition can comprise from about 80% to about 97%, alternatively from about 85% to about 97%, and alternatively from about 90% to about 95% of a solvent, by weight of the oxidizing composition.

The oxidizing composition can further comprise one or more optional ingredients selected from the group consisting of chelants, radical scavengers, thickeners, rheology modifiers, salt, carbonate ion sources, conditioning agents, surfactants, perfumes, and mixtures thereof.

In an embodiment, the method can also be used for hair depilation/removal. Here, the pH of the reducing composition can be from about 8 to about 12, alternatively from about 9 to about 11.5, and alternatively about 10.

The method of treating hair can first comprise separating the hair (which is washed and towel-dried) into multiple sections, and then these sections can be rolled onto curlers (optional for straightening). The curlers used for permanent waves can have a diameter of about 5 mm to about 13 mm, while the curlers used for straightening can have a diameter greater than 13 mm.

After the rolling on curlers is finished, the curlers can be thoroughly wetted down using the required quantity of the reducing composition, which can be from about 60 g to about 120 g.

The amount of time the permanent shaping composition stays on the hair can be from about 1 minute to about 30 minutes, alternatively from about 15 minutes to about 30 minutes. This action time can be shortened by adding heat via the use of a heat radiator or a hood dryer.

After the action time has elapsed that is sufficient for the permanent shaping, which is dependent upon hair quality, the pH value, the shaping effectiveness of the shaping agent, the desired level of change, as well as on the application temperature, the hair is then rinsed with water. Optionally, for straightening, the hair may be dried and then flattened with a heated device such as a flat iron to achieve desired shape.

Thereafter, the hair is oxidatively post-treated (“fixed”). The oxidizing composition can be used in a quantity of from about 50 g to about 200 g, alternatively from about 80 g to about 100 g, depending on hair thickness and length. The concentration of the oxidizing agent can vary depending on application time (normally about 1 minute to about 40 minutes, alternatively about 5 minutes to about 20 minutes) and application temperature (25 deg. C. to 50 deg. C.).

After an action period required for the fixing composition of from about 3 minutes to about 15 minutes, alternatively from about 5 minutes to about 10 minutes, the curlers are removed (if used). It can be advantageous if the hair is then finally shaped as desired and then dried.

### Examples

The following examples illustrate the compositions as described herein. The exemplified compositions may be prepared by conventional formulation and mixing techniques. It will be appreciated that other modifications of the compositions described herein within the skill of those in the art can be undertaken without departing from the spirit and scope of compositions described herein. All parts, percentages, and ratios herein are by weight unless otherwise specified. Some components may come from suppliers as dilute solutions. The amount stated reflects the weight percent of the active material, unless otherwise specified.

### Reducing Compositions

% by weight

Composition A

| | |
|---|---|
| Odorless thiol¹ | 2.5-15 |
| Ammonium Hydroxide (aq. 28% active) | 4.5 |
| Water | qs to 100 |

Composition B

| | |
|---|---|
| Odorless thiol¹ | 2.5-15 |
| Ammonium carbonate | 10 |
| Water | qs to 100 |

Composition C

| | |
|---|---|
| Odorless thiol¹ | 2.5-15 |
| FlexiThix ™² | 5 |
| Phenoxyethanol | 0.3 |
| Sodium Benzoate | 0.2 |
| Disodium EDTA | 0.1 |
| Ammonium Hydroxide (aq. 28% active) | 4 |
| Water | qs to 100 |

Composition D

| | |
|---|---|
| Odorless thiol¹ | 2.5-15 |
| Aculyn ™ 46³ | 15.8 |
| Phenoxyethanol | 0.3 |
| Sodium Benzoate | 0.3 |
| Disodium EDTA | 0.1 |
| Ammonium Hydroxide (aq. 28% active) | 4 |
| Water | qs to 100 |

Composition E

| | |
|---|---|
| Odorless thiol¹ | 2.5-15 |
| Plantaren ® 2000 N UP⁴ | 20 |
| Phenoxyethanol | 0.3 |
| Sodium Benzoate | 0.3 |
| Disodium EDTA | 0.1 |
| Ammonium Hydroxide (aq. 28% active) | 4 |
| Water | qs to 100 |

Composition F

| | |
|---|---|
| Odorless thiol¹ | 2.5-15 |
| Foaming agent | 5 |
| Phenoxyethanol | 0.3 |
| Sodium Benzoate | 0.3 |
| Disodium EDTA | 0.1 |
| Ammonium Hydroxide (aq. 28% active) | 4 |
| Water | qs to 100.0% |

¹The odorless thiol may be any odorless thiol described herein
²PVP polymer supplied by Ashland
³PEG-150/Stearyl/SMDI copolymer supplied by Rohm and Haas
⁴Chemical makeup supplied by BASF

### Oxidizing Compositions

% by weight

Composition G

| | |
|---|---|
| Hydrogen Peroxide | 0.5-12 |
| Water | qs to 100 |

Composition H

| | |
|---|---|
| Hydrogen Peroxide | 3 |
| Cetyl/stearyl alcohol | 4 |
| Salicylic acid | 0.1 |
| Phosphoric acid | 0.09 |
| Etidronic acid | 0.01 |
| Fragrance | 0.4 |
| Water | qs to 100 |

Composition I

| | |
|---|---|
| Hydrogen Peroxide | 2 |
| Salicylic acid | 0.1 |
| Disodium hydrogen phosphate | 0.2 |
| Phosphoric acid | 0.15 |
| Ethoxylated castor oil | 1 |
| Vinylpyrrolidone/styrene copolymer | 0.1 |
| Fragrance | 0.1 |
| Water | qs to 100 |

### Data

### Mannequin Head Odor and Curl Expert Evaluation

Control perm: Omniperm™ with ammonium thioglycolate, available from Zotos Professional, including both the reducing and oxidizing steps.

### Test Perm:

Reducing Composition

| | |
|---|---|
| 3-((2-mercaptoethyl)amino)-1- | 10 |
| methylpyridin-1-ium iodide | |
| Ammonium carbonate | 5 |
| Ammonium hydroxide | to pH = 9.6 |
| Water | qs to 100 |

Oxidizing Composition

| | |
|---|---|
| Hydrogen Peroxide | 3 |
| Cetyl/stearyl alcohol | 4 |
| Salicylic acid | 0.1 |
| Phosphoric acid | 0.09 |
| Etidronic acid | 0.01 |
| Fragrance | 0.4 |
| Water | qs to 100 |

### Odor Assessments:

Treatment and expert sensory performed by licensed cosmetologist with 20+ years expertise in perms. All evaluations shown in graphs were done with hair in wet state, before drying. Sulfur odor was zero from the beginning with the test perm treatment. During treatment, the test perm had some ammonia odor from the ammonium hydroxide/carbonate buffer which is represented in Table 1, “Total Odor.”

TABLE 1
WET HAIR EVALUATIONS:

| | Sulfur Odor | | Total Odor | |
|---|---|---|---|---|
| Scale: | Control | | Control | |
| 0 (none)-5 (most) | Perm | Test Perm | Perm | Test Perm |
| Hair processing | 5 | 0 | 5 | 3 |
| After neutralizing | 5 | 0 | 5 | 1 |
| After 1 wash | 4 | 0 | 4 | 0 |
| After 2 washes | 4 | 0 | 4 | 0 |
| After 3 washes | 3 | 0 | 3 | 0 |
| After 4 washes | 3 | 0 | 3 | 0 |
| After 5 washes | 3 | 0 | 3 | 0 |
| After 10 washes | 1 | 0 | 1 | 0 |
| After 15 washes | 1 | 0 | 1 | 0 |
| After 20 washes | 1 | 0 | 1 | 0 |

### Curl Retention Assessments:

Treatment and expert sensory performed by licensed cosmetologist with 20+ years expertise in perms. All evaluations shown in graphs were done with hair in dry state, after air drying. Photos are available for most time points as well.

TABLE 2
AIR DRIED CURL EVALUATIONS:

Curl Retention
Scale: 0 (none)-5 (most)

| | Control Perm | Test Perm |
|---|---|---|
| After treatment | 5 | 5 |
| After 1 wash | 4 | 4 |
| After 2 washes | 4 | 4 |
| After 3 washes | 4 | 4 |
| After 4 washes | 4 | 4 |
| After 5 washes | 4 | 4 |
| After 10 washes | 4 | 4 |
| After 15 washes | 4 | 4 |
| After 20 washes | 4 | 4 |

### CONCLUSION

3-((2-mercaptoethyl)amino)-1-methylpyridin-1-ium iodide test treatment provided equal curl retention (data in Table 2) through 20 washes to benchmark control perm but with significantly less odor than control (data in Table 1).

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method for treating hair comprising:
a. putting the hair in a desired shape;
b. before and/or after the hair is put in the desired shape, applying a reducing composition to the hair comprising:
i. from about 2.5% to about 15% of a cationic mercapto amino pyridinium compound or a cationic dimercapto amino pyridinium compound, by weight of the reducing composition;
ii. from about 4% to about 10% of a buffering system, by weight of the reducing composition; and
iii. from about 50% to about 93.5% of a solvent, by weight of the reducing composition;
wherein the reducing composition has a pH of from about 8 to about 10.5;
c. rinsing the reducing composition from the hair;
d. applying an oxidizing composition to the hair comprising:
i. from about 0.5% to about 12% of an oxidizing agent, by weight of the oxidizing composition; and
ii. from about 80% to about 97% of a solvent, by weight of the oxidizing composition; and
e. rinsing the oxidizing composition from the hair.

2. The method of claim 1, wherein the reducing composition comprises from about 55% to about 92.5% of the solvent, by weight of the reducing composition.

3. The method of claim 1, wherein the reducing composition comprises from about 60% to about 92.5% of the solvent, by weight of the reducing composition.

4. The method of claim 1, wherein the reducing composition comprises from about 65% to about 92.5% of the solvent, by weight of the reducing composition.

5. The method of claim 1, wherein the reducing composition comprises from about 70% to about 90% of the solvent, by weight of the reducing composition.

6. The method of claim 1, wherein the oxidizing composition comprises from about 85% to about 97% of the solvent, by weight of the oxidizing composition.

7. The method of claim 1, wherein the oxidizing composition comprises from about 90% to about 95% of the solvent, by weight of the oxidizing composition.

8. The method of claim 1, wherein the oxidizing agent is hydrogen peroxide.

9. The method of claim 1, wherein the reducing composition comprises from about 5% to about 13% of a cationic mercapto amino pyridinium compound or a cationic dimercapto amino pyridinium compound, by weight of the reducing composition.

10. The method of claim 1, wherein the reducing composition comprises from about 7% to about 12% of a cationic mercapto amino pyridinium compound or a cationic dimercapto amino pyridinium compound, by weight of the reducing composition.

11. The method of claim 1, wherein the reducing composition comprises from about 9% to about 11% of a cationic mercapto amino pyridinium compound or a cationic dimercapto amino pyridinium compound, by weight of the reducing composition.

12. The method of claim 1, wherein the reducing composition comprises the cationic mercapto amino pyridinium compound.

13. The method of claim 1, wherein the cationic mercapto amino pyridinium compound is 3-((2-mercaptoethyl)amino)-1-methylpyridin-1-ium iodide.

14. The method of claim 1, wherein the reducing composition comprises from about 5% to about 7% of the buffering system, by weight of the reducing composition.

15. The method of claim 1, wherein the reducing composition has a pH of from about 9 to about 10.

16. The method of claim 1, wherein the buffering system comprises ammonium carbonate and ammonium hydroxide.

17. The method of claim 1, wherein the oxidizing composition comprises from about 1% to about 7% of the oxidizing agent, by weight of the oxidizing composition.

18. The method of claim 1, wherein the oxidizing composition comprises from about 1% to about 5% of the oxidizing agent, by weight of the oxidizing composition.

19. The method of claim 1, wherein the reducing composition further comprises one or more optional ingredients selected from the group consisting of chelants, radical scavengers, thickeners, rheology modifiers, salt, carbonate ion sources, conditioning agents, surfactants, perfumes, and mixtures thereof.

20. The method of claim 1, wherein the oxidizing composition further comprises one or more optional ingredients selected from the group consisting of chelants, radical scavengers, thickeners, rheology modifiers, salt, carbonate ion sources, conditioning agents, surfactants, perfumes, and mixtures thereof.
